(19) United States
(12) Patent Application Publication
(10) Pub. No.: US 2002/0062105 A1
(43) Pub. Date: May 23, 2002

(54) Apparatus and method for micro-volume infusion

(76) Inventors: Howard M. C. Tanner, Logan, UT (US); Stephen R. Topaz, St. Helen, OR (US); Royce Herbst, Alpine, UT (US); David N. Jones, Salt Lake City, UT (US)

Correspondence Address: TRASKBRITT, PC, P.O. BOX 2550, Salt Lake City, UT, 84110, US

(21) Appl. No.: 09/963,021

(22) Filed: Sep. 25, 2001

Related U.S. Application Data
(60) Provisional application No. 60/234,742, filed on Sep. 22, 2000.

(51) Int. Cl. A61M 37/00

(52) U.S. Cl. 604/131; 264/478

(57) ABSTRACT

An apparatus and method for volumetric micro-liter infusion of therapeutic liquids. This portable pump system remains unobtrusive and less restrictive of patient ambulation and orientation. The present invention includes expendables, the disposability of which facilitates safe reuse of more expensive portions and is sufficiently simple for use in a home care environment. It may be designed to incorporate real-time, interactive or remote monitoring and regulation and visual or audible indication of system pressures, chemical balances and other important variables.

### CLAIM OF PRIORITY

Pursuant to the provisions of 35 U.S.C. 119(e), this application claims the benefit of the filing date of provisional patent application Serial No. 60/234,742, filed Sep. 22, 2000, for “APPARATUS AND METHOD FOR MICRO-VOLUME INFUSION.”

### BACKGROUND OF THE INVENTION

1. Field of the Invention

This invention relates to fluid transfer. It is particularly directed to an improved system for volumetric micro-liter infusion of therapeutic liquids to a selected treatment site of a medical patient.

2. State of the Art

Medical practice has numerous devices for fluid transfer involving metered micro-doses of therapeutic liquids. Early paradigms for micro-volumetric fluid transfers involved drain-feed metering structure such as those disclosed in U.S. Pat. No. 3,887,428 to Seagle et al., entitled “Variable Infusion Control Device,” and U.S. Pat. No. 5,234,413 to Wonder et al., entitled “Infusion Rate Regulator Device” These structures presuppose expensive extraneous support from peripheral equipment and personnel. Restricted mobility of the patient is also a disadvantage.

Such disadvantages have catalyzed more recent paradigms involving pumps, many of which pumps have the disadvantage of substantial costs to own and use. Responsive to recognized shortcomings, pumps have evolved to become portable and lower cost. Some pumps utilize disposable single-patient components. Illustrative of such pumps are those disclosed in U.S. Pat. No. 5,632,606 to Jacobsen et al., entitled “Volumetric Pump/Valve,” and its progeny.

Some peristaltic pumps have been utilized for low volume fluid transport, though the pressure rollers of such pumps are characteristically mounted to rotate on an axis that is transverse the plane on which they revolve. The term “plane” or “plane of revolution,” as used in this disclosure, refers to a reference plane oriented to intersect the midpoints of the pressure rollers of the pump. Shortcomings associated with such prior art mounting of peristaltic pump rollers include the requirement for more space, as well as a weaker operational framework.

Similarly, nonplanar prior art peristaltic mechanisms utilize flow paths formed of autonomous collapsible conduit, and accordingly are subject to the attendant disadvantages of conduit that is susceptible to entanglement, crimping and disconnection.

Heretofore, peristaltic pumps have typically incorporated free standing fluid conduit cassettes with numerous parts, necessitating assembly with its attendant costs.

Other existing systems suffer from a number of disadvantages. One disadvantage of piston-type pump mechanisms is their discontinuous flow patterns. A defined bolus of liquid is infused at each piston cycle. Transfer of lower volumes of liquid is achieved by programming piston cycles to occur less frequently. The average volume of liquid transferred is low. However, this “average” is achieved with intermittent bursts of liquid, rather than with a continuous low micro-liter flow rate.

Another disadvantage of prior art pumps is difficulty in programming the pumps or in designating preselected programs. Limited manual dexterity, fading eye sight, diminished strength, reduced cognitive function and other problems normally associated with conditions of ill health or aging are common among users of these outpatient pumps, necessitating simple, facile operation of the pump. Yet prior art pumps are characteristically programmed and manipulated with small, difficult to grasp knobs labeled with minute print and presented in configurations that to some patients may be difficult to understand.

Heretofore, inefficiencies have resulted from infusion of excessive or insufficient amounts of therapeutic liquid relative to the needs of the particular patient during a particular time frame, such as in the case of total perenteral nutrition or analgesics. Similarly, chemotherapy or other medicaments infused at generalized rates and concentrations may rather arbitrarily, yet erroneously, be presumed to be well suited to the physiology of the particular patient, with little or no direct, on-line or real-time corroboration of the patient's responsiveness to treatment.

### SUMMARY OF THE INVENTION

The present invention provides an improved apparatus and method for volumetric micro-liter fluid transfer. Various embodiments of the invention provide significant benefits, notably: a pump system capable of providing continuous volumetric micro-liter flow rates; a pump system with single patient components that are reliable yet inexpensive, disposable and sufficiently simple for use in a home care or other ambulatory environment; a pump system wherein electronic circuitry and associated indicator systems enable monitoring and regulation of therapeutic liquid relative to patient physiology status, flow rates, system pressure status, system pressure change, system pressure change rates, pressure status, change and change rates at selected anatomical sites, temperatures, system timing, chemical balances and other therapeutic variables to mimic more closely and/or optimize normal physiological patterns and to enhance the patient's and physician's ability to determine optimal dosage, regime and protocol to obtain such goals; such monitoring and regulation optionally from locations remote from the patient, and whether remote or not, the foregoing features capable of being actuated automatically or by direct intervention based on real time interaction; a peristaltic pump mechanism with rollers whose axes are parallel with the plane of revolution; a peristaltic pump mechanism with a flow path integrally formed in the surrounding members; and a method of manufacturing a fluid conduit integrally formed with surrounding members for use with a peristaltic mechanism.

A typical portable peristaltic pump system of this invention, applied to volumetric micro-liter infusion of therapeutic fluid, includes a motor, which may be structured and arranged to rotate at approximately 5,500 rpm, a drive train, which may include a gear assembly capable of reducing the rpm's of the motor to about 20 rpm's, and a roller housing associated with and driven by the motor through the drive train.

The roller housing typically comprises a plurality of rollers, each roller having an exterior circumference that includes a pinch surface and each roller structured and arranged to rotate around a respective roller axis. A housing axis may be included around which the roller housing can revolve on a plane. The plane in one embodiment may be substantially parallel with respect to each respective roller axis. Consistent with the foregoing, each respective roller axis may be structured and arranged to be other than perpendicular with respect to the plane. In a related embodiment, the pinch surface of each respective roller axis may be along at least one position of rotation substantially parallel with respect to the plane.

The system further comprises a disposable cartridge interface, which may include a cassette path formed of polyurethane or other flexible medical grade flat stock wherein is formed a cavity configuration, and may also including a collapsible fluid conduit. The cavity configuration may be structured and arranged to include the collapsible fluid conduit. In this embodiment, the fluid conduit may include an upstream end and a downstream end and may be integrally formed as a part of a flexible cassette path with a periphery, the majority of which may be circumscribed by a rigid or semi-rigid member. This semi-rigid member may advantageously be formed of a polystyrene material.

A typical system further includes a drive plate structured and arranged to abut a first side of the fluid conduit as the pinch surface of each respective roller presses against a second side of the fluid conduit while the roller housing revolves. In this arrangement, fluid may be transferred from a source into the fluid conduit through the upstream end and then out the downstream end to a selected destination.

The disposable cartridge interface, in one preferred embodiment, may comprise a collapsible fluid conduit, including an upstream conduit, a peristaltic interface in fluid communication with the upstream conduit and a downstream conduit in fluid communication with the peristaltic interface. The peristaltic interface in this embodiment comprises a plurality of interface channels, each including an upstream end. At least one valve is located near the upstream end of at least one of those interface channels. The interface further includes means whereby that valve may be operated to open, thereby to admit fluid from the upstream conduit through the upstream end of the interface channel or to close, thereby to prevent fluid from the upstream conduit through the upstream end of the interface channel. This valve operation thus selectively increases or decreases fluid throughput through the plurality of interface channels.

In a typical practical embodiment, operation of the valve(s) to open the interface channel(s) to fluid flow may increase volume throughput capacity up to 1 liter per hour, whereas operation of any valve(s) in the system to close all but one of the interface channels to fluid flow may decrease volume throughput capacity to as low as about 0.05 ml per hour. In this embodiment, a drive plate may be structured and arranged to abut a first side of the fluid conduit as the pinch surface of each respective roller presses against a second side of the fluid conduit along a substantial portion of the peristaltic interface housed between the first side and second side while the roller housing revolves.

The present invention in its preferred embodiment, further contemplates a method of manufacturing a mask work embodying an integral cavity configuration. The method comprises several steps, the first of which is to provide a mold plate assembly including a first face in which a desired cavity configuration with a periphery is formed, a second face for abutting the first face and at least one access point whereby negative or positive pressure from a source external to the mold plate may be applied to the cavity configuration. The second step involves providing a sheet of flexible polymer flat stock with a first exposure and a second exposure facing a direction opposite the first exposure, placing at least a first portion of the flat stock between the first side and second side of the mold plate assembly with the first exposure abutting the first face and anchoring the first side and second side together with the first portion of flat stock remaining indwelling between the first side and second side. A further step involves associating the mold plate assembly including the indwelling first portion of flat stock with the external pressure source and then heating the mold plate assembly and the indwelling first portion of flat stock to a degree sufficient to surmount the memory of the flat stock. The steps, while the flat stock is in this heated condition, involve applying selectively negative or positive pressure to the cavity configuration in sufficient degree to displace a part of the flat stock specifically into the cavity configuration and then cooling the flat stock to a degree sufficient to establish the memory of the part of the flat stock displaced into and in conformity with the cavity configuration, such that the first exposure of the first portion of the flat stock is formed to include a convex configuration corresponding to the cavity configuration and the second exposure is formed to include a concave configuration corresponding to the cavity configuration.

Continuing, the first portion of the flat stock is removed from between the mold plate assembly and a second portion of the flat stock is folded against the first portion such that the second exposure of the first portion is opposite to and lays against the second exposure of the second portion. Finally, the first portion and second portion are bonded together in close proximity to the periphery of the cavity configuration. The bonding may be accomplished by a dielectric weld, which may form a continuous seal along the entire periphery of the cavity configuration. The cavity configuration may thus be arranged to define a fluid conduit for use in a cassette path of a disposable cartridge interface of a peristaltic pump. The cavity configuration may further comprise at least one compartment structured and arranged to accommodate a fluid metering device and at least one compartment structured and arranged to accommodate a pressure sensor. The pressure sensor may be structured and arranged to communicate interactively with the pump in response and relative to information provided by the pressure sensor and any preprogramming of the pump.

In a further enhancement, the fluid flow path defined by the bonding may include an upstream fluid opening and a downstream fluid opening wherein the first portion and second portion are bonded together at or near an outer edge of the flat stock to define a bag portion space between the first exposure and second exposure. This space is in communication with the upstream fluid opening but is sealed against fluid flow in any other direction.

The pump system of one presently preferred embodiment may comprise a pump mechanism; a fluid conduit comprising an upstream conduit, a downstream conduit and an interface conduit associated with the pump mechanism for pumping fluid. In this embodiment, a fluid metering system in fluid communication with the downstream conduit is included and comprises a plurality of valves. It further comprises a means of governing actuation of the plurality of valves in accordance with an information signal. A fluid restrictor stage includes a plurality of possible fluid flow paths. Any one or combination of these fluid paths may be selectively accessed or bypassed in accordance with actuation of any or all of the plurality of the valves subject to the actuation governing means. Fluid flow may be further reduced to preselected micro-liter flow rates substantially lower than rates of flow achievable by the pump mechanism alone. A pressure sensor may be associated with the downstream conduit upstream from the fluid restrictor stage, whereby pressure characteristics of the fluid in the downstream conduit upstream from the fluid restrictor stage may be ascertained to a more subtle degree thereby enabling inference of pressure characteristics of the fluid in the downstream conduit downstream from the fluid restrictor stage, said pressure sensor being in communication with the governing means.

The pressure characteristics may include pressure status, pressure change, rate of pressure change, or any or all of the foregoing, whereby the pressure sensor may communicate pressure characteristics to the governing means as a basis for the governing means to govern and coordinate actuation of the plurality of valves for achievement of optimal pump system performance in accordance with predetermined specifications.

A signal processing feedback loop may be associated with the downstream conduit. The feedback loop may comprise a means of governing the rpm rate of the pump mechanism in accordance with an information signal. The feedback loop may further comprise a pressure transducer that is in communication with the pump governing means and that is capable of ascertaining pressure characteristics of the fluid within the downstream conduit and capable of supplying to the pump governing means the information signal pertaining to ascertained pressure characteristics.

The pump governing means and the actuation governing means may be coordinated based upon the information signal to achieve either dynamic valving or dynamic metering or both dynamic valving and dynamic metering of fluid flow within the downstream conduit. The pressure transducer may be a strain gauge capable of sensing mechanical movement by means of changes in an electrical signal from a micro circuit.

Yet another embodiment of the present novel advance is a portable acoustic streaming pump system for volumetric micro-liter infusion of therapeutic fluid. This system may include a fluid conduit comprising an upstream conduit, a downstream conduit and an interface conduit. It may further comprise a fluid chamber in communication with the fluid conduit and a piezoelectric material wafer associated with the fluid chamber and in communication with a source of electric current, whereby electric current may be introduced to the piezoelectric material to cause a deflection of the wafer repeatedly resulting in a fluid flow within the conduit. In this system, the fluid flow may be in communication with a pressure sensor or fluid metering means or both a pressure sensor and fluid metering means to achieve greater consistency and definition in dynamic fluid flow.

### BRIEF DESCRIPTION OF THE SEVERAL VIEWS OF THE DRAWINGS

In the drawings, which illustrate what is currently regarded as the best mode for carrying out the invention:

FIG. 1 is a partially exploded perspective view of a portion of a preferred embodiment of the invention;

FIG. 2 illustrates a partially exploded view of a subassembly of the invention;

FIG. 3 presents a cross-sectional view of a partially assembled embodiment of a portion of a pump;

FIG. 4a is a perspective view of the top of a cassette path;

FIG. 4b is a perspective view of the bottom of the cassette path of FIG. 4a;

FIG. 5 portrays a perspective view of a cassette frame;

FIG. 6 depicts a partial cut-away top view of the fluid metering and pressure sensing mechanisms;

FIG. 7 sets forth a schematic of the circuitry associating a strain gauge and amplifier with an analog to digital converter, computer and variable motor; and

### DETAILED DESCRIPTION OF THE ILLUSTRATED EMBODIMENT

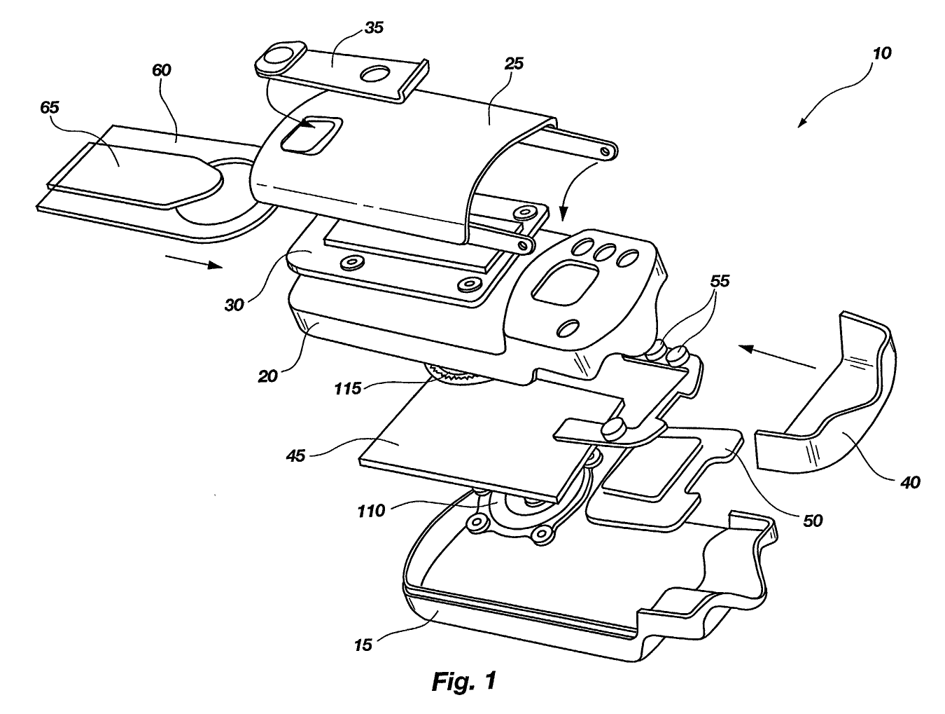

The structural elements of one configuration of the present novel device system for liquid transfer are presented in FIG. 1. A pump 10 comprises a case base 15, a case top 20, a hinging lid 25, a lid liner 30, a lid catch 35, a battery access door 40, an electronics PCB 45, a control PCB 50, a membrane switch panel 55 and a cassette frame 60 associated with a cassette path 65.

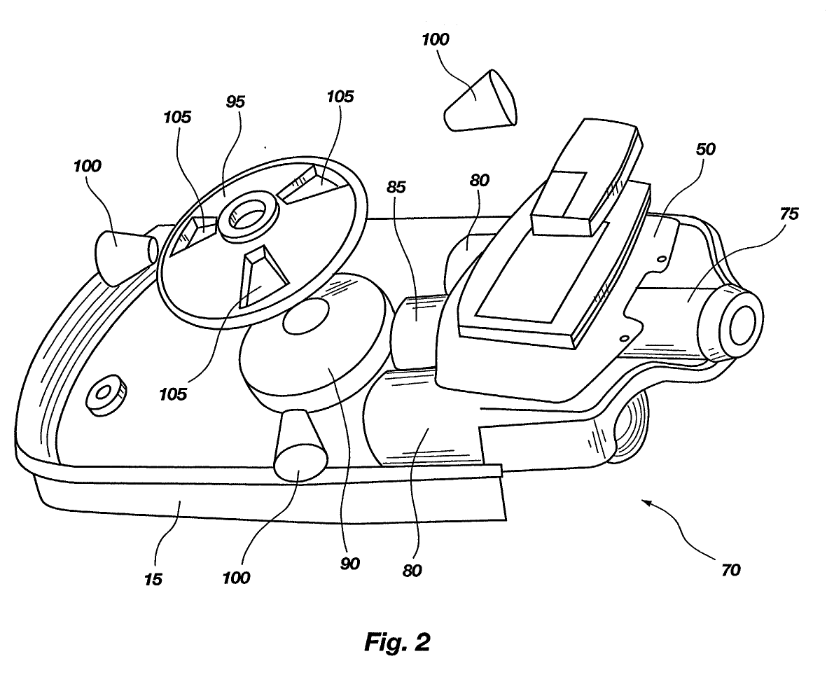

While any of a variety of pump mechanisms may be incorporated in the pump 10, such as for example screw, scroll, piezoelectric acoustic streaming, WankelÔ type, gear pump and the like, FIG. 2 more particularly sets forth a peristaltic mechanism 70. The peristaltic mechanism 70 in the present embodiment is associated with the case base 15 and comprises essentially a motor 75, batteries 80, a drive plate 85, a drive train 90, a roller case 95 and rollers 100.

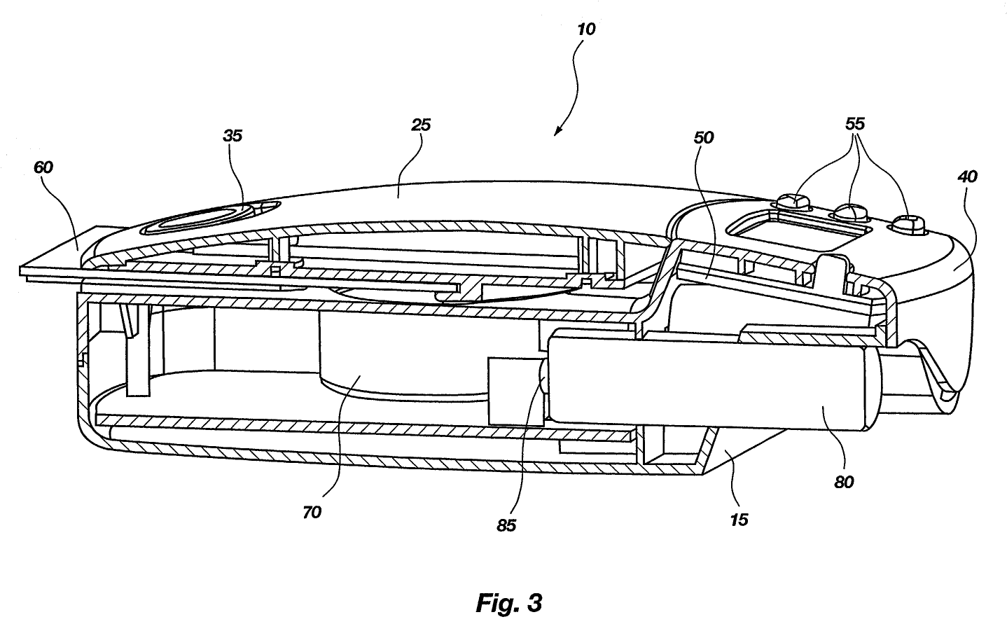

When assembled as illustrated generally in FIG. 3, the rollers 100, which may be spherical, conical, cylindrical or some other suitable shape, are situated within corresponding roller spaces 105 formed in the roller case 95, and may be mounted on bearings, an axle or the like (not shown). The roller case 95 is associated with the roller housing 110 depicted in FIG. 1. The motor 75 is associated with appropriate gears engaged with the drive train 85 at a ratio that reduces operational rpm from 5,500 to about 30, to achieve the desired range of fluid flow rates.

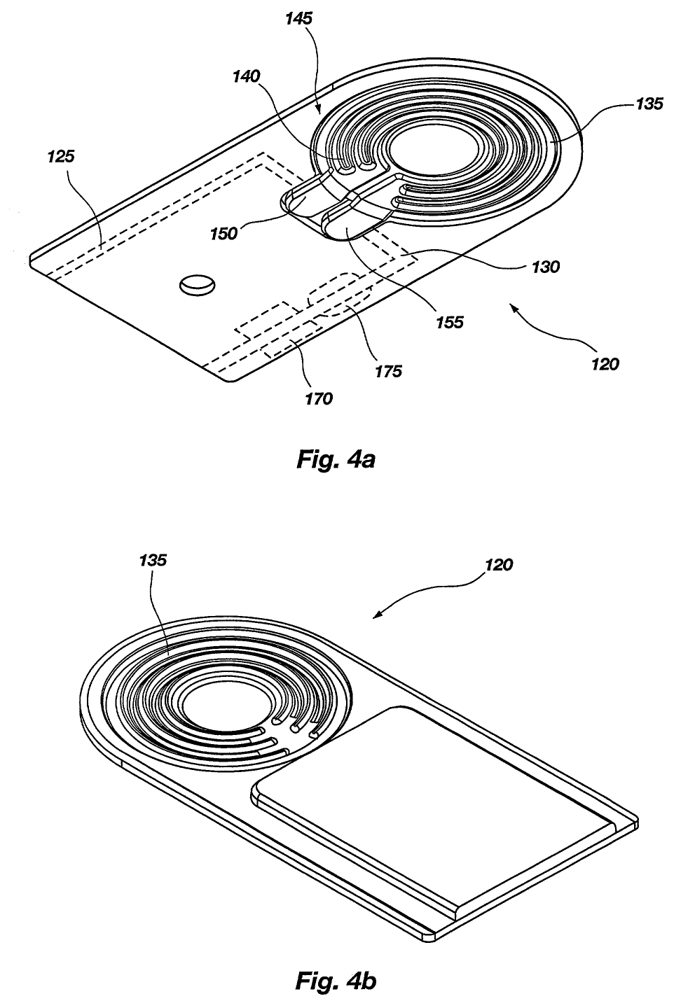

FIGS. 4a and 4b portray the cassette path 120, from a top and bottom perspective, respectively. The cassette path 120 is preferably formed of two part pressure or vacuum formed polyurethane, though any strong, flexible, inert, inexpensive and disposable material may be used. The path 120 includes an upstream conduit 125 and a downstream conduit 130 which communicate with each other through at least one, but preferably a plurality of peristaltic channels 135. The rollers 100 in operation traverse any and all peristaltic channels 135 as they revolve, compressing such channels 135 against the drive plate 90.

In configurations involving two or more such channels 135, channel valving 140 may be incorporated preferably at the upstream head 145 of all of the peristaltic channels 135 or of at least all but one of the peristaltic channels 135 to enable a user to selectively increase or decrease the number of channels through which liquid may be directed, thereby to augment or diminish the potential capacity of the pump 10 for liquid throughput. Accordingly, the total cross-sectional dimension of each of the upstream and downstream conduits 125, 130 should be sufficiently sized to accommodate the total throughput capacity of the peristaltic channels 135 when all channel valving 140 is open, for maximum flow.

Thus, even as the motor 75 is driven at its lowest possible rpm, yet further reductions in flow rate may be achieved by reduction of the number of peristaltic channels 135 utilized. Yet further reductions may be accomplished by utilization of successively smaller cross-sectional diameters among the plurality of peristaltic channels 135, with the smallest such diameter being embodied in the peristaltic channel 135 that alone remains open when all others of the plurality of the peristaltic channels 135 are selectively closed by the channel valving 140.

In operation, fluid flows from a reservoir source (not shown) into the upstream conduit 125 and into one or more of the peristaltic channels 135. Where more than one peristaltic channel 135 is open, fluid being pumped by the pump 10 is fed into all open peristaltic channels 135 from an upstream pool 150 where the fluid is pooled after passing through the upstream conduit 125. A downstream pool 155 performs a converse function by pooling all fluid that has passed through two or more of the peristaltic channels 135 prior to communication of the fluid through the downstream conduit 130. The need for a pooling function and channel valving 140 is of course obviated in configurations utilizing only a single peristaltic channel 135.

The cassette path 120 may optionally be integrally molded with the reservoir source (not shown). Such reservoir sources typically have a liquid volume capacity of 250 ml (cc) for micro-liter infusion applications.

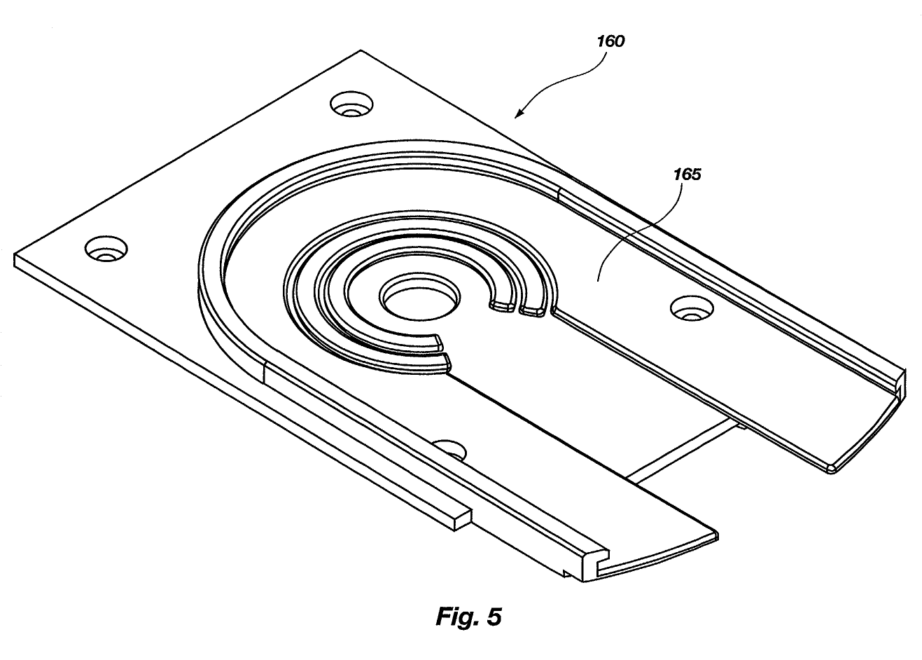

The cassette path 120 of the presently preferred embodiment is associated with a cassette frame 160, illustrated in FIG. 5, which may be made of a rigid or semi-rigid material such as polystyrene. The cassette path 120 may be so associated with the cassette frame 160 by being anchored within the cassette site 165 by a clamp fit or sonic welding or other conventional means of attachment.

With the present advance in the art, it may readily be discerned that any of a variety of compartments may be directly configured into the cassette path 120. One such compartment may, for example, accommodate a novel fluid metering system 170.

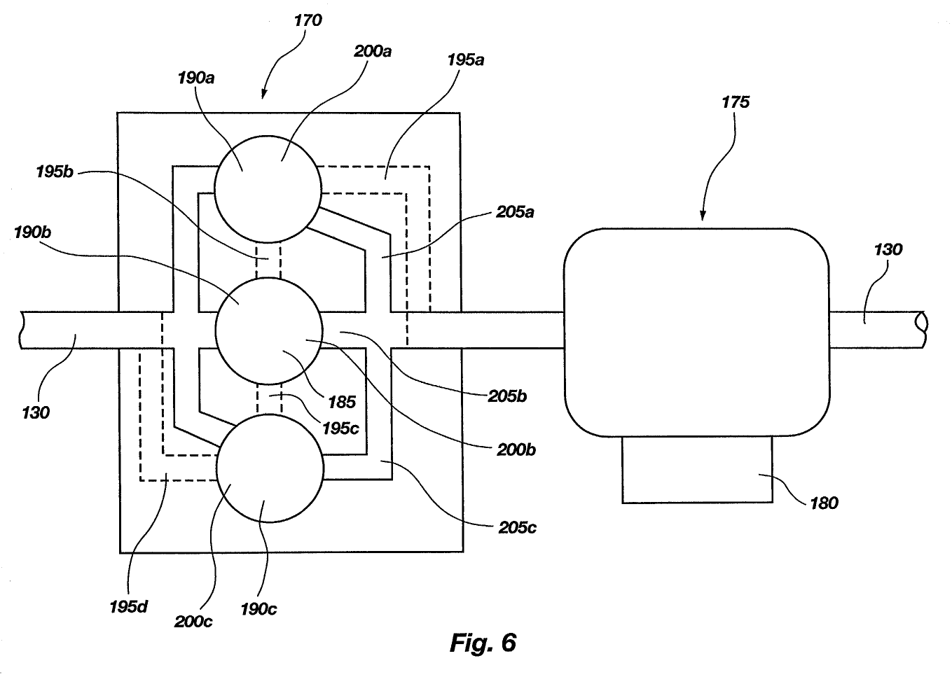

The fluid metering system 170 in the present embodiment, illustrated in FIG. 6, is in fluid communication with the downstream conduit 130 downstream from a pressure sensor 175, also accommodated within a compartment configured into the cassette path 120. The pressure sensor 175 may involve a strain gauge, piezoelectric or other suitable micro-structure.

The strain gauge 180 of the present embodiment is in communication with a computer (not shown) or other electronic aspect with monitoring and regulating capability and participates actively in the sensing of pressure upstream from the fluid metering system 170 and downstream from the peristaltic mechanism 70 of the pump 10.

The fluid metering system 170 may utilize any fluid restricting means, but is presently embodied as a fluid restrictor 185. The fluid restrictor 185, which may be formed of a suitable polymer such as polypropylene, acetate or MylarÔ of an ester type, comprises a predetermined porosity to achieve a given degree of restriction. Alternatively, the fluid metering system 170 may utilize a series of fluid restrictors 190a, 190b, 190c that direct fluid by a series valving system (not shown) through a series of conduits 195a, 195b, 195c, 195d (designated with dashes). Yet another alternative, partially illustrated, may involve a plurality of restrictors 200a, 200b, 200c, to which fluid may directed through a plurality of conduits 205a, 205b, 205c in varying combinations and permutations achieved by a permuting valving system (not shown).

Use of fluid restrictors of varying porosity in the series 190 or plurality 200 restrictors enable further refinements to the fluid restriction function. These various configurations each enable varying and subtle degrees of fluid restriction to be designed into the overall system function. The upstream conduit 125 and downstream conduit 130 may be embodied in a single multi-lumen structure (not shown) accessing the body of a medical patient and may further selectively comprise additional lumen or lumens for further diagnostic or therapeutic capabilities vis-à-vis the patient. Thus, when a liquid is infused to the vascular or other system of a medical patient, for example, this novel fluid metering system 170 accordingly enables detection of subtle pressure differentials, changes and rates of change. Such detection may be at the point of access of the body or alternatively simultaneously at multiple selected sites within the body.

Whereas fluid metering may be grossly accomplished by varying the speed of the motor 75 alone, with the enhanced dynamics introduced by the interplay between the pressure sensor 175 and the fluid restrictor 185, particularly when combined with refined flow rates accomplished with the selective variation in number of peristaltic channels utilized in the peristaltic mechanism 70, micro-liter volumetric infusion of therapeutic liquids is enabled in the present embodiment with greater specificity, continuity, reliability and commensurate efficacy and safety than has heretofore been achievable.

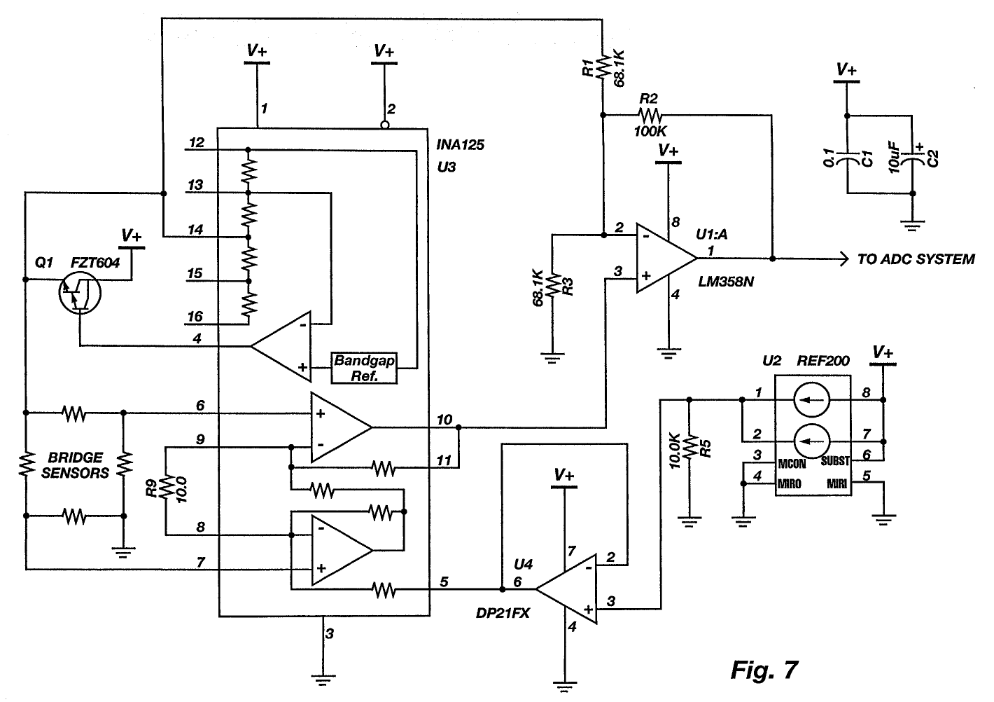

More particularly set forth in FIG. 7 is a signal processing feedback loop. A strain gauge amplifier provides feedback signal so that the roller pump controller may establish the targeted fluid pressure at the roller pump exit chamber. Pressure developed upstream from the flow resistance or restriction stage is sensed with a pressure sensor which may be a strain gauge, piezoelectric or similar micro-circuit structure such as a four resistive-element strain gauge bridge. Minute differential changes of resistance within the bridge result from changes in the mechanical force applied onto the strain gauge “beam.” These resistance changes are detected and converted to a resultant output voltage by integrated circuit U1. U1 also provides a reference voltage used to maintain constant voltage across the strain gauge elements.

U1, a Burr-Brown INA 125, is chosen for this application due to its low power consumption and its ability to operate from a single ended supply. The device is specified to operate over a positive supply range from +2.7 to +36 volts. In operational mode, the amplifier draws a maximum of 525 uAmps. This rating does not include the current drawn by the strain gauge elements. One embodiment of the device maintains 2.5 volts across the 120 ohm strain gauge network yielding a bridge excitation current of 20.8 mA, several orders of magnitude larger than the amplifier drain. Transistor Q1 acts as a current booster within the feedback loop to allow the bridge to be driven at this relatively high current. The reference voltage is programmed as a multiple (×1,×2,×4,or ×8) of the 1.24 internal reference by closing the feedback loop at the appropriate point (U1 pin 14 in this case).

Amplifier U1 is interposed between the pressure sensor and an analog to digital converter to amplify the signal preparatory to operation of a computer stage which governs a variable motor drive in accordance with read-only memory (ROM) software in the computer. The amplifier may be placed in “sleep mode” to reduce its power consumption by pulling pin 2 to a low logic level (<=+0.1 volts). This reduces current drain to +/−1 uAmp (typ) +/−25 uAmp (max). The INA 125 data sheet provides that in one embodiment during “sleep mode” the reference voltage may shut down across the bridge. In the hand-wired prototype, U1 pin 2 is connected directly to the positive supply rail.

A precision current reference U2 and resistor R2 create a “local ground” for the output stage of U1. This brings the bridge inputs (approximately 1.25 volts at pins 6 and 7) within the allowable common mode range of the amplifier's input section. A follower stage provides this reference voltage to U1 pin 5 to prevent degradation of CMRR due to source impedance.

Differential gain of U1 is programmed by resistor R1 according to the formula G=4+(60000/R1) where R1 is specified in ohms.

Operational amplifier U3 provides gain and level shifting to bring the output of U1 into a range more appropriate for data conversion.

Depending on the particular design to be implemented at the system level, the number of parts required to embody the foregoing might be reduced if a bipolar power supply were available. For example, U2,U4 and associated components could be eliminated.

To reduce the likelihood of malfunction resulting from use of a measurement system utilizing more than one reference, a single reference may be utilized by both the ADC and for bridge excitation to enable reference errors due to initial tolerance, temperature drift, long term stability and the like to “gang up on each other” through use of ratiometric techniques.

The allowable input range, the useful output swing, and the operation of the internal voltage reference are constrained by the power supply voltage, particularly in a single-ended, low voltage battery application.

The device system and method of the present invention provide distinct advantages over prior peritoneal dialysis systems and methods. Thus, reference herein to specific details of the illustrated or other preferred embodiments is by way of example and not intended to limit the scope of the appended claims. It will be apparent to those skilled in the art that many modifications of the basic illustrated embodiments may be made without departing from the spirit and scope of the invention as recited by the claims.

What is claimed is:

1. A portable peristaltic pump system for volumetric micro-liter infusion of therapeutic fluid, including:
a motor;
a drive train;
a roller housing associated with and driven by said motor through said drive train, said roller housing comprising:
a plurality of rollers, each roller structured and arranged to rotate around a respective roller axis; and
a housing axis around which said roller housing is mounted to revolve on a plane;
wherein said plane is substantially parallel with respect to each said respective roller axis;
a disposable cartridge interface, comprising a collapsible fluid conduit including an upstream end and a downstream end; and
a drive plate structured and arranged to abut a first side of said fluid conduit as each respective roller presses against a second side of said fluid conduit while the roller housing revolves;
whereby fluid may be transferred from a source into said fluid conduit through said upstream end and then out said downstream end to a destination.

2. The pump system of claim 1, further comprising a gear assembly associated with said drive train and capable of reducing the rpm's of said motor.

3. The pump system of claim 2, wherein said motor rotates at approximately 5,500 rpm and said gear assembly is structured and arranged to reduce the rpm's of said motor to about 20 rpm's.

4. A portable peristaltic pump system for volumetric micro-liter infusion of
therapeutic liquid, including:
a motor which rotates at approximately 5,500 rpm;
a drive train, including a gear assembly capable of reducing the rpm's of said motor to about 20 rpm's;
a roller housing associated with and driven by said motor through said drive train, said roller housing comprising:
a plurality of rollers, each roller structured and arranged to rotate around a respective roller axis; and
a housing axis around which said roller housing can revolve on a plane;
wherein each respective roller axis is not perpendicular with respect to said plane;
a disposable cartridge interface, comprising a collapsible fluid conduit including an upstream end and a downstream end; and
a drive plate structured and arranged to abut a first side of said fluid conduit as each respective roller presses against a second side of said fluid conduit while said roller housing revolves;
whereby fluid may be transferred from a source into said fluid conduit through said upstream end and then out said downstream end to a destination.

5. The pump system of claim 4, wherein said collapsible fluid conduit is integrally formed as a part of a flexible cassette path with a periphery.

6. The pump system of claim 5, wherein said disposable cartridge interface further comprises a rigid or semi-rigid member circumscribing a majority of said periphery of said flexible cassette path.

7. The pump system of claim 5, wherein said flexible cassette path is formed of a polyurethane material.

8. The pump system of claim 6, wherein said rigid or semi-rigid member is formed of a polystyrene material.

9. A portable peristaltic pump system for volumetric micro-liter infusion of
therapeutic liquid, including:
a motor which rotates at approximately 5,500 rpm;
a drive train, including a gear assembly capable of reducing the rpm's of said motor to about 20 rpm's;
a roller housing associated with and driven by said motor through said drive train, said roller housing comprising:
a plurality of rollers, the exterior circumference of each said roller including a pinch surface and each said roller structured and arranged to rotate around a respective roller axis; and
a housing axis around which said roller housing can revolve on a plane;
wherein said pinch surface of each respective roller axis is along at least one position of rotation substantially parallel with respect to said plane;
a disposable cartridge interface, comprising a collapsible fluid conduit including an upstream end and a downstream end; and
a drive plate structured and arranged to abut a first side of said fluid conduit as said pinch surface of each respective roller presses against a second side of said fluid conduit while said roller housing revolves;
whereby fluid may be transferred from a source into said fluid conduit through said upstream end and then out said downstream end to a destination.

10. A method of manufacturing a mask work embodying an integral cavity configuration, comprising the steps of:
providing a mold plate assembly comprising a first face in which a desired cavity configuration with a periphery is formed, a second face for abutting said first face and at least one access point whereby negative or positive pressure from a source external to said mold plate may be applied to said cavity configuration;
providing a sheet of flexible polymer flat stock with a first exposure and a second exposure facing a direction opposite said first exposure;
placing at least a first portion of said flat stock between said first side and said second side of said mold plate assembly with said first exposure abutting said first face and anchoring said first side and said second side together with said first portion of flat stock remaining indwelling between said first side and said second side;
associating said mold plate assembly including said indwelling first portion of flat stock with said external pressure source;
heating said mold plate assembly and said indwelling first portion of flat stock to a degree sufficient to surmount a first memory characteristic of said flat stock;
applying selectively negative or positive pressure to said cavity configuration in sufficient degree to displace a part of said flat stock specifically into said cavity configuration;
cooling said flat stock to a degree sufficient to establish a second memory characteristic of said part of said flat stock displaced into and in conformity with said cavity configuration, such that said first exposure of said first portion of said flat stock is formed to include a convex configuration corresponding to said cavity configuration and said second exposure is formed to include a concave configuration corresponding to said cavity configuration;
removing said first portion of said flat stock from between said mold plate assembly and folding a second portion of said flat stock against said first portion such that said second exposure of said first portion is opposite to and lays against said second exposure of said second portion; and
bonding said first portion and said second portion together in close proximity to said periphery of said cavity configuration.

11. The method of claim 10, wherein said bonding is accomplished by a dielectric weld.

12. The method of claim 11, wherein said dielectric weld forms a continuous seal along said entire periphery of said cavity configuration.

13. The method of claim 12, wherein said cavity configuration defines a fluid conduit for use in a cassette path of a disposable cartridge interface of a peristaltic pump.

14. The method of claim 13, wherein said cavity configuration further comprises at least one compartment structured and arranged to accommodate a fluid metering device.

15. The method of claim 13, wherein said cavity configuration further comprises at least one compartment structured and arranged to accommodate a pressure sensor.

16. The method of claim 15, wherein said pressure sensor is structured and arranged to communicate interactively with said pump in response and relative to information provided by said pressure sensor and any preprogramming of said pump.

17. The method of claim 10, wherein said bonding is accomplished with a dielectric weld and forms a continuous seal along said entire periphery of said cavity configuration to define a fluid flow path, said flow path including an upstream fluid opening and a downstream fluid opening, and wherein said bonding further comprises bonding said first portion and said second portion together at or near an outer edge of said flat stock to define a bag portion space between said first exposure and second exposure that is in communication with said upstream fluid opening but sealed against fluid flow in any other direction.

18. A portable peristaltic pump system for volumetric micro-liter infusion of
therapeutic fluid, including:
a motor which rotates at approximately 5,500 rpm;
a drive train, including a gear assembly capable of reducing the rpm's of said motor to about 20 rpm's;
a roller housing associated with and driven by said motor through said drive train, said roller housing comprising:
a plurality of rollers, said exterior circumference of each roller including a pinch surface and each roller structured and arranged to rotate around a respective roller axis; and
a housing axis around which said roller housing can revolve on a plane;
wherein said pinch surface of each respective roller axis is along at least one position of rotation substantially not perpendicular with respect to said plane;
a disposable cartridge interface, comprising:
a cassette path formed of polyurethane or other flexible medical grade flat stock wherein is formed a cavity configuration comprising a collapsible fluid conduit between an upstream inlet a downstream outlet, said conduit compressible by said pinch surface of each roller as each roller rolls across said conduit; and
a drive plate structured and arranged to abut a first side of said fluid conduit as said pinch surface of each respective roller presses against a second side of said fluid conduit while said roller housing revolves;
whereby fluid may be transferred from a source into said fluid conduit through said upstream end and then out said downstream end to a destination.

19. A portable pump system for volumetric micro-liter infusion of therapeutic fluid, including:
a pump mechanism;
a fluid conduit comprising an upstream conduit, a downstream conduit and an interface conduit associated with said pump mechanism for pumping fluid; and
a fluid metering system in fluid communication with said downstream conduit and comprising:
a plurality of valves;
a means of governing actuation of said plurality of valves in accordance with an information signal; and
a fluid restrictor stage including a plurality of possible fluid flow paths any one or combination or all of which may be selectively accessed or bypassed in accordance with actuation of any or all of said plurality of said valves subject to said actuation governing means.

20. The pump system of claim 19, whereby fluid flow may be further reduced to preselected micro-liter flow rates substantially lower than rates of flow achievable by said pump mechanism alone.

21. The pump system of claim 19, wherein a pressure sensor is associated with said downstream conduit upstream from said fluid restrictor stage, whereby pressure characteristics of said fluid in said downstream conduit upstream from said fluid restrictor stage may be ascertained to a more subtle degree thereby enabling inference of pressure characteristics of said fluid in said downstream conduit downstream from said fluid restrictor stage, said pressure sensor being in communication with said governing means.

22. The pump system of claim 21, wherein said pressure characteristics may include:
pressure status;
pressure change;
rate of pressure change; or
any or all of the foregoing;
whereby said pressure sensor may communicate pressure characteristics to said governing means as a basis for said governing means to govern and coordinate actuation of said plurality of valves for achievement of optimal pump system performance in accordance with predetermined specifications.

23. A portable pump system for volumetric micro-liter infusion of therapeutic fluid, including:
a pump mechanism;
a fluid conduit comprising an upstream conduit, a downstream conduit and an interface conduit associated with said pump mechanism for pumping fluid; and
a signal processing feedback loop associated with said downstream conduit and comprising:
a means of governing the rpm rate of said pump mechanism in accordance with an information signal; and
a pressure transducer in communication with said pump governing means,
capable of ascertaining pressure characteristics of said fluid within said downstream conduit, and
capable of supplying to said pump governing means said information signal pertaining to ascertained pressure characteristics.

24. The pump system of claim 23, whereby said pump governing means and said actuation governing means are coordinated based upon said information signal to achieve either dynamic valving or dynamic metering or both dynamic valving and dynamic metering of fluid flow within said downstream conduit.

25. The pump system of claim 24, wherein said pressure transducer is a strain gauge capable of sensing mechanical movement by means of changes in an electrical signal from a micro circuit.

26. A portable peristaltic pump system for volumetric micro-liter infusion of therapeutic liquid, including:
a motor which rotates at approximately 5,500 rpm;
a drive train, including a gear assembly capable of reducing the rpm's of said motor to about 20 rpm's;
a roller housing associated with and driven by said motor through said drive train, said roller housing comprising:
a plurality of rollers, said exterior circumference of each roller including a pinch surface and each roller structured and arranged to rotate around a respective roller axis; and
a housing axis around which said roller housing can revolve on a plane;
wherein said pinch surface of each respective roller axis is along at least one position of rotation substantially parallel with respect to said plane;
a disposable cartridge interface, comprising:
a collapsible fluid conduit, including an upstream conduit, a peristaltic interface in fluid communication with said upstream conduit and a downstream conduit in fluid communication with said peristaltic interface;
said peristaltic interface comprising a plurality of interface channels each including:
an upstream end,
at least one valve located near said upstream end of at least one of said plurality of interface channels, and
means whereby said at least one valve may be operated to open and thereby admit fluid from said upstream conduit through said upstream end of at least one interface channel or to close and thereby prevent fluid from said upstream conduit through said upstream end of at least one interface channel, thereby to respectively increase or decrease fluid throughput through said plurality of interface channels;
a drive plate structured and arranged to abut a first side of said fluid conduit as said pinch surface of each respective roller presses against a second side of said fluid conduit along a substantial portion of said peristaltic interface housed between said first side and second side while said roller housing revolves;
whereby fluid may be transferred from a source into said fluid conduit through said upstream end and then out said downstream end to a destination.

27. Said pump system of claim 26, wherein operation of said at least one valve to open said at least one interface channel to fluid flow may increase volume throughput capacity up to 1 liter per hour.

28. Said pump system of claim 26, wherein operation of all of said at least one valve to close all but one of said plurality of interface channels to fluid flow may decrease volume throughput capacity to about 0.05 ml per hour.

29. A portable acoustic streaming pump system for volumetric micro-liter infusion of therapeutic fluid, including:
a fluid conduit comprising an upstream conduit, a downstream conduit and an interface conduit;
a fluid chamber in communication with said fluid conduit; and
a piezoelectric material wafer associated with said fluid chamber and in communication with a source of electric current;
whereby electric current may be introduced to said piezoelectric material to cause a deflection of said wafer repeatedly resulting in a fluid flow within said conduit.

30. Said pump system of claim 29, wherein said fluid flow may be in communication with a pressure sensor or fluid metering means or both a pressure sensor and fluid metering means to achieve greater consistency and definition in dynamic fluid flow.
